(19) United States
(12) Patent Application Publication
(10) Pub. No.: US 2006/0210644 A1
(43) Pub. Date: Sep. 21, 2006

(54) Materials, methods, and devices for treatment of arthropathies and spondylopathies

(76) Inventor: Bruce Levin, Philadelphia, PA (US)

Correspondence Address: KENYON & KENYON LLP, ONE BROADWAY, NEW YORK, NY, 10004, US

(21) Appl. No.: 11/303,509

(22) Filed: Dec. 16, 2005

Related U.S. Application Data
(60) Provisional application No. 60/636,901, filed on Dec. 16, 2004.

(51) Int. Cl. A61K 33/00

(52) U.S. Cl. 424/600

(57) ABSTRACT

Novel modalities are introduced to treat joint and cartilage ischemia and related pathologies to improve outcome in the treatment of arthropathies and spondylopathies. The invention includes compositions, materials or devices which will improve oxygen, substrate and nutrient delivery to joint tissues and modalities to decrease the degradation of joint tissues by inflammatory and other destructive processes.

### FIELD OF THE INVENTION

The invention provides novel methods for treating joint and cartilage ischemia and related pathologies. The methods are useful to achieve improved outcomes in the treatment of arthropathies and spondylopathies. The invention includes compositions, materials and devices that improve oxygen, substrate, and nutrient delivery to joint tissues.

### BACKGROUND OF THE INVENTION

Degenerative joint and disc diseases are very prevalent in all countries and are responsible for causing millions of patients significant and often severe pain and varying degrees of disability. The economic and other costs of these disorders are staggering; lost wages, medically necessary care, and other disease associated phenomena bleed billions of dollars from the global community and negatively impact numerous people.

Arthritis is primarily a disease of the joint complex most specifically targeting cartilage. This tissue component exists in at least five subtypes and is synthesized very slowly by chondrocytes. Episodes, or even a single remote episode, of trauma to a joint predisposes it to develop arthritis which may become symptomatic years later. Chronic repetitive mechanical microtrauma, or overuse or overstrain of joints is well recognized as a major risk factor in the development of degenerative joint and spine diseases. Elite athletes seem to be at particular risk even when they are relatively young. Initially, osteoarthritis was in fact felt to be purely a disease of “wear and tear.” Now it is very well accepted that a significant inflammatory pathophysiologic component is involved in its genesis and progression.

While much is known about many of the pathophysiologic processes which contribute to arthropathic and degenerative intervertebral disc disease entities, there are significant gaps in current theories which in turn severely limit the abilities of those fluent in the current art to design and implement more effective therapeutic modalities. Many pharmaceutical and nutriceutical agents have been developed, but the definitive cure for these diseases is currently surgical, often involving excision of the joint and replacement with an artificial mechanical joint or even fusion in extreme cases. These options are suboptimal as artificial joints cannot replicate normal joint function and have significant attendant complications, limited lifetimes and are financially burdensome. There remains an unmet need for therapies which are useful for the preservation or restoration of joint or disc function, physiology and structure.

Hence, many research initiatives have been undertaken to find and develop anti-inflammatory compounds. Arthritic joints have high local levels of metalloproteases and other tissue destructive enzymes, autocoids, prostaglandins, cytokines, TNF, and other inflammatory mediators, and patients with arthritis have high blood levels of markers of inflammation such as protein C and are at increased statistical risk of developing other systemic diseases with inflammatory components, including cardiovascular disease. Current research emphasizes the development of anti-inflammatory agents which may be able to delay the development or progression of arthritis. NSAIDS, COX-2, and 5-LOX inhibitors have been used with only limited success. However, results remain disappointing.

Nutriceuticals, including sulfur donor compounds such as methyl-sulfonyl-methane (MSM), glutathione and cysteine which are thought to provide sulfur to cartilage producing cells, have been suggested to be helpful, but are not scientifically recognized as having any significant clinical efficacy. Chondroitin sulfate and glucosamine have been utilized to treat arthritis and some studies seem to suggest that they do have some degree of efficacy. It is felt that they may provide some precursor supply, although this is not certain. Similarly intrarticular Hyaline products, such as Synvisc, may have some short term efficacy. Similarly, a variety of vitamins, minerals and other compounds have been suggested by the lay press to be potentially helpful, but there is no evidence that in non deficiency states that they are.

Cartilage transplants, which may be autologous or non autologous have proven generally ineffective thus far. The longevity of the transplanted cartilage or chondrocytes seems to be short. Furthermore, adherence to the articular complex is poor and structural integration is also disappointing. Hence, introducing healthy cartilage cells or material into a pathologic environment is suboptimal in therapeutic terms.

### SUMMARY OF THE INVENTION

The inventor, while not being limited by them, herein discloses key points and concepts which better define and explain important components of the pathophysiologic processes involved in the pathogenesis of arthropathies, including degenerative joint diseases such as osteoarthritis, and related soft tissue and cartilage diseases affecting structure and function of important cartilaginous, collagenous, soft tissue, osseous and other structures. As a result of a better understanding of these novel concepts, more effective therapeutic modalities were developed and are herein disclosed.

### DETAILED DESCRIPTION OF THE INVENTION

Arthropathies are joint diseases and include diseases of the bone as well as diseases of soft tissue. Arthropathies include, but are not limited to osteoarthropathy as well as infectious joint disease. Spondylopathies are diseases of the vertebrae or spinal column. The invention relates to novel treatments of arthropathies and spondylopathies. More particularly, the invention relates to novel treatments for degenerative diseases of bone and soft tissue, and particularly to joint and disc diseases.

Cartilage is highly avascular tissue. Cartilage and associated cellular elements therefore receive oxygen and required nutrients and necessary substrates from diffusion, osmosis, as well as other active and passive transport mechanisms and related processes. These processes require the traversal of significant distances from the feeding vascular structures. A degree of normal joint mobility is felt to provide mechanical assistance to the ingress of oxygen and nutrients and the egress of deleterious substance including waste products of normal and abnormal metabolic processes. Likely, mechanic strain or trauma to a joint results in a degree of violation of the joint blood barrier complex. This results in an inflammatory response which consists of cellular and humeral immunologic limbs. While inflammation is a major component of the disease complex, other co pathologies are also critical. For example, increased systemic or local concentrations of precursor or other requisite compounds and cofactors may shift the synthetic equilibrium forward, favoring the production of healthy cartilage.

A key pathologic process distinct from increasing precursor delivery plays a critical role. It is herein disclosed that a key contributor to the pathophysiology which defines arthritic and related processes is a significant borderline ischemic component. It is well recognized that poor weather often exacerbates arthritis symptoms. It is postulated that this phenomenon is due to increased inflammatory activity in response to changing barometric pressures, and that there is also a pressure/temperature dependent component which affects the delivery of oxygen and nutrients to joint tissues. If the barometric pressure is low, the forward driving pressure gradient is adversely affected. Because the nature of the arteriolar microcirculation differs from that of the venous side microcirculation, particularly across a non homogenous avascular and edematous inflamed tissue field there is no complementary significant distal effective negative driving pressure to compensate for this. The net result is that less oxygen and nutrients are pushed forward into the cartilage and ischemic joint tissues.

It is also likely that there is an ideal local temperature range which affects macro and microcirculation as well as local active and passive transport mechanisms. A too cold temperature would increase vasoconstriction, blood viscosity, and adversely affect oxygen release from hemoglobin, but decrease the immunological cellular and enzymatic limbs of the inflammatory response. A high temperature may result in maximal vasodilation and oxygen release from hemoglobin, with attendant increased oxygen delivery, but the inflammatory mechanisms may be increased to the point that oxygen consumption is increased locally.

The following experiment was undertaken which provides evidence of some degree of a borderline hypoxic or ischemic component of arthritis pathophysiology.

A middle aged male with degenerative joint disease of both hips noted significant worsening of symptoms preceding and during rainy weather. On three separate occasions, during poor weather with low barometric pressures, treatment with oxygen by mask or nasal cannulae for 4 to 12 hours decreased his symptoms during these periods. It is recognized in the surgical literature that administration nasal oxygen postoperatively to patients undergoing bowel surgery aids in the healing of the postoperative bowel and is associated with a decreased incidence of complications. This occurs despite a very modest increase in blood oxygen saturation levels.

The inventor therefore postulates a novel approach to intervening against what appear to be plausible pathophysiologic processes which are responsible for the degenerative arthropathies and related diseases.

The pathogenesis of arthritic diseases and related disorders is certainly complex. As increased mechanical stress is placed on a joint, wear and tear is increased and cartilage is damaged. Lost cartilage is replaced by chondrocytes and the chemical and structural characteristics of the cartilage are dependent upon the type of chondrocyte, location of the joint, availability of required metabolic and synthetic substrates, influencing physical factors such as strain or pressure, and cellular and humoral mediators as well as the age of the joint and the presence of systemic diseases. As the cartilage matrix is degraded over a period of time the joint swells and the collagen and cartilage produced is inferior and has less affinity to adhere water molecules. As the ingress and egress of cartilage bound water molecules is a major contributing component towards the ability of cartilage to absorb shock, mechanical stress on the joint is greatly increased. While joint space dimensions appear preserved on imaging studies such as x rays, CT scans or MRI scans, the articular cartilage and joint are severely compromised. Only recently have MRI parameters become available which assess actual cartilage damage and non gross articular defects. Thus, arthritis is often diagnostically confirmed later in its natural course in many patients.

As noted earlier, as mechanical stress increases on the joint, the joint-blood barrier complex is compromised in certain areas. As is seen with violation of the blood-brain barrier, blood-eye barrier, or blood-testicle barrier, immunosensitization occurs with antigenic substances being released into the systemic circulation and locally. For example, severe trauma to one eye may make it necessary to remove the injured eye in a timely fashion to prevent an immunologic and destructive response against the uninjured eye. The greater the antigenic challenge, the greater the immunologic response over a wide range. The antigenic challenge may vary with tissue type, location, inherent antigenicity and amount of antigen delivered across compromised barrier areas. These autoimmune/immune responses may vary in severity, specificity, location, and nature. In arthritis, an increased humoral and cellular inflammatory mediator response engenders significant tissue inflammation and cartilage destruction. Indeed, a wide variety of proteases and destructive enzymes are found in diseased joints, and their inactivation or dilution decreases arthritis symptoms.

As inflammatory changes persist, the inventor believes oxygen and nutrient/substrate delivery to chondrocytes, other cells, cartilage and joint tissues is decreased by several mechanisms. Firstly, swelling and edema increase the distance between the oxygen/nutrient/substrate rich tissues and structures and the chondrocytes, cartilage and other oxygen/nutrient starved joint elements. Furthermore, tissue swelling and edema increase tissue pressures which decrease forward driving arterial microvascular hydrostatic and other forces, which favor the forward transport of oxygen/nutrients/substrates from the arteriolar sided capillary microvasculature towards the relatively ischemic cells and tissues. Next, adhesions, chronically deposited materials, and byproducts of inflammation form an additional physical and no physiologic barrier against oxygen/nutrient diffusion and transport. The normal anatomic and functional microvascular relationships are altered such that delivery-requirement balances become mismatched.

Increased venous pooling is commonly found in and around inflamed tissues, and altered venous micro and macro structures and altered physiology create increased venous backpressure which has a net affect of decreasing arteriolar forward driving pressure gradients. Microcirculatory sub structural and permeability changes also favor the egress of cellular and humoral mediators of inflammation at the expense of normal delivery of oxygen/nutrients. Furthermore, a “traffic jam” of inflammatory cells locally decreases the absolute numbers of red cells locally. These inflammatory cells also use up locally available oxygen. This, combined with the other attendant metabolic costs of ongoing inflammation further depletes local delivery and availability of oxygen in the borderline ischemic joint and in other structures.

As cartilage is destroyed, the dynamic compliance of the joint changes and periods of altered joint geometry occur, and tethering ligaments and other structures become lax, which introduces increasing lateral instability and other strains on the joint. This, in turn, further increases cartilage destruction.

Also very damaging to cartilage are the changes in subchondral bone. As mechanical stress increases on the joint, the subchondral bone changes from a sponge like and compliant soft bone to a thicker, denser sclerotic non porous and noncompliant bone. The loss of the mechanical buffering and shock absorbing qualities of normal subchondral bone places further mechanical stress on the cartilage, further increasing cartilage destruction. It is thought that the sclerotic bone forms a barrier which decreases effective diffusion, transport and delivery of oxygen/nutrients/substrates to the cartilage. This, combined with decreased delivery of oxygen/nutrients/substrates from inflamed synovial, bursal, and other structures, results in chronic and changing patterns of watershed type ischemia.

Chronic low grade or high grade regional ischemic insult leads to defective synthesis of articular collagen and cartilage while symptoms of arthritis may emerge at a later time. Furthermore, as the tissues become ischemic, there is increased release of products of ischemia with attendant increased inflammatory response processes. This is quite tissue destructive and also costly in metabolic terms. For example, a tourniquet applied to a limb to render it non bloody during certain orthopedic or other surgical procedures renders it ischemic for a period of time. When the tourniquet is released and circulation is restored, a washout of the products of limb ischemia occurs. Severe hypotension, and bradycardia or tachycardia can occur, and the patient may develop adult respiratory distress syndrome, coagulapathies, or systemic inflammatory response syndrome. A similar but much worse syndrome occurred during early attempts at surgical transplant of donor livers to patients with end stage liver disease. When the blood supply to the transplanted liver was established, the patient often sustained severe hypotension, dysrhthmias and even death. Flushing the liver preoperatively with two liters of normal saline to remove accumulated metabolites seems to have played a major role in decreasing the morbidity and mortality of liver transplants. Such acute systemic inflammatory responses to severe acute episodes of tissue ischemia are easily recognized. However, chronic low grade ischemia is not commonly recognized nor clinically appreciated.

Thus, a chronic low grade ischemic state of the joint complex contributes to the development of low grade catabolic and inflammatory responses which contribute to the disease process. This may explain, in part, the failure of other therapies to significantly alter the progress of the disease. For example, anti-inflammatory agents may be helpful, and may even help restore some degree of normal joint function and perfusion, but in the setting of chronic inflammation they cannot reverse the process. An analogous situation was seen in ulcer disease where antacids were helpful but often noncurative, until it was discovered that H. pylori infection played a key pathogenic role in many patients who were effectively treated with antibiotics. Similarly, chondroitin sulfate, glucosamine and other synthetic precursors cannot be expected to be incorporated into cartilage properly in an inflammatory ischemic environment. The pathologic replication of chondrocytes into clusters of multiple chondrocytes seen in arthritic cartilage may represent attempts to make up for the inferior quality of arthritic cartilage secondary to impaired synthesis. This of course places genetic strain on the tissue as the number of divisions is predetermined or otherwise limited by telomere length and other factors contributing to cell senescence.

Based on this disease model, novel modalities for the treatment of arthropathies, arthritis, disc disease and related pathologies are provided herein. These modalities may be utilized individually or in combination, with or without other known modalities. According to the invention, improved treatment of degenerative bone and soft tissue disesase of the joints and spine are comprise delivery of oxygen to the affected tissue. The methods of the invention further provide for delivery of nutrients and other substances to the diseased tissue, as well as removal or inactivation of damaging agents such as cytokines and inflammatory precursors.

Methods and materials to provide oxygen and/or nutrients or other required substrates may range from the simple to more complex. One way to increased oxygen availability is to increase oxygen transport and delivery to the tissue to be treated. In one embodiment, systemic oxygen concentration is increased. For example, supplemental oxygen may delivered to the lungs by any means known to one skilled in the art. In an embodiment of the invention, the subject is provided with a respiratory atmosphere that has increased oxygen content. In another embodiment, a hyperbaric chambers can be utilized. Such therapies are useful to increase circulating hemoglobin bound oxygen or dissolved oxygen. Other manipulations known that allow increased oxygen dissociation from hemoglobin at the treatment site. One such manipulation is to manipulate red blood cell 2,3 diphosphoglycerate (2.3 DPG) or introducing a fetal type of hemoglobin. In other embodiments, blood substitutes such as perflurocarbon compounds or free hemoglobin or other blood substitutes may be useful.

Systemic oxygen availability can also be improved by increasing blood flow to the tissue to be treated. Drugs which can increase joint perfusion may be helpful. Drugs which decrease viscosity or aid in the more efficient flow of red cells in the microcirculation may be therapeutic.

Direct or indirect introduction of oxygen or nutrient or substrate containing substances may be accomplished by continuous or intermittent perfusion or intermittent delivery directly to the joint, synovium, cartilage, bone, subchondral bone, bone marrow, or any anatomically related structures. In one embodiment a catheter type system is introduced to the appropriate anatomical structure. Perfused fluids can carry oxygen, peroxides, ozone, free hemoglobin or other oxygen carrier agents. In certain embodiments, the perfused fluid is a perfluorocarbon. The system can be open or closed and can contain an oxygenator, filter, pump or any other device known to one skilled in the art of perfusion or circulatory bypass devices. The system can have an ingress and egress component to allow perfusion. It may or may not be designed to be entirely implanted, as is seen in spinal cord medication delivery devices and related devices with a reservoir system.

The delivery tube, or catheter, consists of a distal and proximal end and at least one lumen and may be of any shape or size. It may be constructed from any material known to one skilled in the art, including biological tissues such as cultured artificial vascular strictures or carbon, or other, microtubules. It is ideally inert, nonirritating, atraumatic and may require integral structural components to maintain lumen patency. The distal end may have one or more orifices. It may be retractable, sheathed, or rotatory or have other mechanisms required to maintain orifice patency in the setting of tissue reaction or inflammation. It may be coated, impregnated, or otherwise bound with antibiotics, silver, chemotherapeutic or other agents to prevent infection and decrease tissue reaction. It may be electrically conductive; it may contain or be constructed to allow for housing of biosensors, monitors, lasers, or other electrical equipment or components. Solutions or materials introduced may include gases, gels, solids fluids, liquids, oxygen, air or other gas or compound any oxygenateable substrate, any nutrient including amino acids, any energy substrate, proteins, vitamins, minerals, carbohydrate, fatty acids, lipids, sugars, cartilage precursors, hyaline compounds, anti-inflammatory agents, antibiotics buffers, monoclonal antibodies, growth factors and any compound or substance with efficacious properties. In one embodiment, the joint access device could be similar to a portacath or other related known vascular or other access device with one or more ports. It could be used to access a non articulating or any area of a joint, or placed in the bone, marrow, or related joint structure. This may be surgically or nonsurgically implanted, with the access port subcutaneous or exposed. In addition to allowing the delivery of therapeutic substances at predetermined intervals, these devices would allow for safer joint lavage to dilute destructive enzymes, or other damaging compounds or cells. Examples of devices useful for the present invention include, but are not limited to implantable catheters such as Portacath, dialysis cathaters, injection ports and infusion ports, and lavage systems. Infusaport was developed for occasional blood draws, administration of blood products, chemotherapy, or other drugs. The “port” is a metal or plastic device with a diaphragm on the top that is placed in the fat under the skin and is anchored to the underlying muscle. The port is used by placing a special needle through the overlying skin: the needle has a hole on the side so that the outlet of the needle is not blocked by the back of the port. Lavage systems may be recirculating, allowing introdution of oxygen or other oxygen carrying matrix, and filtration to remove undesired agents such as enzymes, complement, inflammatory cytokines (e.g., IL-6, TNF-α), prostaglandins, and the like. The devices can be manually operated. Alternatively, the devices can be automated and capable of delivering measured doses of oxygen and/or nutrient carrying reagents. Delivery can be continuous or intermittent.

Treatments, whether systemic or direct, that increase oxygen availability at the disease site can be combined with other treatments known in the art for arthropathies, spondylopathies, and related diseases. For example, anti-inflammatory agents or other supplements commonly administered for treatment of arthropathies, arthritis, or disc diseases can be administered. Such agents can be administered systemically or directly. Although it is preferable to administer such agents or supplements together with, and by the same route as treatments that increase oxygen availability, the treatments can also be administered separately.

Direct injection of depot anti-inflammatory agents other than steroids into or around joint structures is novel and will increase efficacy and decrease systemic side effects. As depot steroids contain alchohols, phenols, binders and other irritating or otherwise damaging substances, compositions using less toxic and less irritating compounds are novel and may include liposomes, microsomes and the like. In another embodiment of this invention, depot compositions, pellets, microsomes, liposomes, meshes, carbon microtubules, or other modalities for chronic drug delivery known to one skilled in the art could be introduced into the joint and used to supply or generate oxygen, nutrients, free radical scavengers, anti-inflammatory agents and the like into the local environment decreasing ischemia and inflammation in the cartilage and joint tissues. These compositions, materials, or devices may be formulated or produced to be pressure or temperature sensitive or responsive or to be sensitive or responsive to pH, CO₂, or the presence of inflammatory mediators or ischemic byproducts in order to increase drug or oxygen delivery when inflammation or ischemia is worsening. Similarly, compositions, materials or devices which could absorb, adsorb or inactivate destructive cellular, immunological, or chemical destructive chemicals or materials would be therapeutic.

A shunt type of device can be implanted in the joint, bone marrow or other joint related structure to allow for the egress of harmful metabolites or other destructive compounds or to prevent high pressures in the joint, or in the bone marrow. Sclerotic bone may need to be excised, and possibly MRI or CT reconstruction could guide construction of a suitable patient specific geometric correct biomatrix or other matrix for replacement. This may also guide matrices for joint transplant or replacement. It may be possible to abrade away or otherwise remove portions of the sclerotic bone, or use ultrasound, radiofrequency, LASER or other modalities to make the subchondral bone porous and more compliant and less impenetrable. This would decrease mechanical stress on the joint and may allow for easier oxygen/nutrient transport. Perhaps a cushioning layer or loci of shock absorbing materials such as cartilage, resilin or any suitable substance could be used to allow the bone to heal without fracturing. Subchondral bone cysts could be aspirated or destroyed with any catheter or other surgical modality known to one in the art with introduction of bone graft, matrix or other materials. Perhaps cartilage transplants would be more effective in an oxygenated environment. Perhaps plugs of autologous or heterologous chondrocytes, with or without gene or telomere manipulation, embryonic or stem cells, Wharton's jelly cells or marrow cells could be transplanted or microtransplanted into selected areas and begin normal function.

In an embodiment of the invention, treatment of an arthropathy, spondylopathy, or related disease to reduce ischemia is accompanied by support of the affected tissue. Placing an affected joint for periods in a neutral position or a position where vector forces against physically compromised or defective cartilage may decrease the rate of destruction and allow better defect repair. For example, iliac support may decrease hip pressure as may hip flexion support cushion devices. Tilt Inversion tables are helpful in destressing the spinal column and hip joints, but are suboptimal because excessive forces acting along the inclination angle and not along the plane of supporting structures stress joints and the spinal column and because of excessive strain on the ankles which are the only areas secured. A modified table which supports the patients normal lordosis and kyphosis with cushioning or molded design, or provides hip flexion cushioning, and has multiple options to secure the patient to the table and at several locations, would decrease strain and increase efficacy.

Accordingly, the invention includes treatment modalities that combine tissue support and perfusion or lavage of affected tissue. For example, in a treatment for a disk injury involving traction, or surgical intervention, the affected tissue is perfused or lavaged in such a manner as to reduce ischemia, and optionally to provide other nutrients or stimulants and prevent inflammation. An automated perfusion system can be used at the bedside. A portable perfusion system can be used to continue treatment once the patient is no longer immobilized. Such perfusion or lavage therapy can also be used with immobilized or otherwise supported joint injuries, including injuries to bone and soft tissue. Such combination therapy can be especially valuable where injured tissues are poorly vascularized. For example, owing to poor vascularization, fractures or breaks of certain wrist and ankle bones require extended immobilization that would be significantly reduce if the healing process was accelerated.

It is understood and expected that variations in the principles of the invention herein disclosed may be made by one skilled in the art and it is intended that such modifications are to be included within the scope of the present invention.

What is claimed is:

1. A method of treating a subject having a degenerative disease of joint or spine comprising administering a treatment that increases oxygen availability at the site of the disease.

2. The method of claim 1, wherein the degenerative disease is arthritis.

3. The method of claim 1, wherein the degenerative disease involves an intervertebral disk.

4. The method of claim 1, wherein systemic oxygen concentration is increased in the subject.

5. The method of claim 1, wherein systemic oxygen delivery to the disease is increased.

6. The method of claim 1, wherein oxygen is delivered directly to the site of the disease.

7. The method of claim 6, wherein oxygen is delivered by perfusion of a fluid carrying oxygen, peroxide, ozone, or hemoglobin.

8. The method of claim 6, wherein oxygen is delivered by a catheter or infusion port.

9. The method of claim 6, wherein oxygen is delivered by means of a recirculating fluid from which a harmful agent is filtered.

10. The method of claim 9, where the harmful agent is an inflammatory agent.

11. The method of claim 1, which further comprises delivery of a nutrient to the site of the disease.

12. The method of claim 1, which further comprises delivery of an anti-inflammatory agent to the site of the disease.

13. The method of claim 12, wherein the anti-inflammatory agent is selected from the group consisting of an NSAID, a COX-2 inhibitors, and a 5-LOX inhibitor.

14. The method of claim 1, which further comprises immobilization of the tissue affected by the disease.

15. The method of claim 1, which further comprises physical support of the tissue affected by the disease.
